SEQUENCE LISTING (10 sequences)

SEQ ID NO: 1 - protein, 11 aa, synthetic construct
RASQDVNTAVA

SEQ ID NO: 2 - protein, 7 aa, synthetic construct
SASFLYS

SEQ ID NO: 3 - protein, 9 aa, synthetic construct
QQHYTTPPT

SEQ ID NO: 4 - protein, 5 aa, synthetic construct
DTYIH

SEQ ID NO: 5 - protein, 17 aa, synthetic construct
RIYPTNGYTRYADSVKG

SEQ ID NO: 6 - protein, 11 aa, synthetic construct
WGGDGFYAMDY

SEQ ID NO: 7 - protein, 107 aa, synthetic construct [sequence omitted]

SEQ ID NO: 8 - protein, 120 aa, synthetic construct [sequence omitted]

SEQ ID NO: 9 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0242512 A1
(43) Pub. Date: Aug. 20, 2026

(54) USE OF ANTI-HER2 ANTIBODY IN PREPARATION OF DRUG FOR TREATING CANCER

(71) Applicant: SHENGHE (CHINA) BIOPHARMACEUTICAL CO., LTD., Nanjing, Jiangsu (CN)

(72) Inventors: Tie XU, Nanjing, Jiangsu (CN); Liusong YIN, Nanjing, Jiangsu (CN); Xiaoling JIANG, Nanjing, Jiangsu (CN)

(73) Assignee: SHENGHE (CHINA) BIOPHARMACEUTICAL CO., LTD., Nanjing, Jiangsu (CN)

(21) Appl. No.: 19/160,936

(22) PCT Filed: Mar. 1, 2024
(86) PCT No.: PCT/CN2024/079489

§ 371 Date: Aug. 29, 2025

(30) Foreign Application Priority Data
Mar. 3, 2023 (WO) PCT/CN2023/079432

(51) Int. Cl. C07K 16/32; A61K 39/00; A61P 35/00

(52) U.S. Cl. CPC C07K 16/32; A61P 35/00; A61K 2039/505; A61K 2039/545; C07K 2317/24; C07K 2317/41; C07K 2317/565

(57) ABSTRACT

The use of an anti-HER2 antibody in the preparation of a drug for treating biliary system cancer, gastric cancer, or breast cancer. The anti-HER2 antibody of the embodiments is completely defucosylated, effectively enhancing the affinity of the antibody for the Fc receptor CD16a, improving the ADCC activity of the antibody, and exhibiting excellent anti-tumor activity as well as good safety and tolerability.

### TECHNICAL FIELD

The invention belongs to the field of biomedicine and specifically relates to the use of an anti-HER2 antibody in the preparation of a drug for treating biliary system cancer, gastric cancer, or breast cancer.

### BACKGROUND

HER2 is an abbreviation for human epidermal growth factor receptor 2, which is a proto-oncogene located in region 21 of the long arm of chromosome 17. The transmembrane protein encoding a molecular weight of 185 KD, also known as p185HER2, is a growth factor receptor with transmembrane tyrosine kinase activity. The HER family consists of four members (HER1, HER2, HER3, HER4), all of which are transmembrane tyrosine kinase receptors with growth stimulating activity. HER2 does not have a natural ligand, but can be activated through overexpression of homodimers or heterodimers of other members of the HER family that can be activated by ligand binding, thereby activating receptor tyrosine kinases. Multiple signaling pathways, including mitogen activated protein kinase, phosphatidylinositol-3-kinase protein kinase B/Akt, phospholipase C-protein kinase C, and transcription signal transduction and activation proteins, trigger downstream signal cascade reactions. HER2 is expressed at low levels in a very small number of tissues, while it is overexpressed in over 30% of human tumors. The overexpression of HER2 gene is not only associated with the occurrence and development of tumors, but also an important clinical treatment detection and prognostic indicator, and an important target for the selection of tumor targeted therapy drugs.

At present, the best targeted drug for the treatment of HER2 positive breast cancer is Roche's anti-HER2 monoclonal antibody (trastuzumab, trade name Herceptin®), which has become the first line drug for the treatment of breast cancer at home and abroad. The research on the treatment of HER2 positive breast cancer with trastuzumab published at present shows that after an average of four years of treatment observation, nearly 90% of female patients receiving treatment still survive, and show good cardiac safety and tolerance.

The most important genetic polymorphism of IgG Fc receptor is CD16a, which can be classified into three genotypes based on whether the amino acid at position 158 is phenylalanine (Phe, F) or valine (Val, V): 158V/V, 158V/F, and 158F/F. The clinical response of patients to trastuzumab is closely related to the polymorphism of CD16a in patients. The ORR ratios of trastuzumab in patients with three genotypes were CD16a 158 V/V (82%), 158 V/F (42%), and 158 F/F (35%), respectively, with significant differences. In the treatment with trastuzumab, the progression free survival of CD16a 158 V/V, 158 V/F, and 158 F/F genotype patients was 15.0 months, 11.1 months, and 12.9 months, respectively. The genetic polymorphism of Fc receptors, their PBMC mediated ADCC activity, and the clinical response of patients to trastuzumab are consistent. Trastuzumab cannot effectively mediate ADCC activity in CD16a 158 F/F type patients, which is also a reason for the ineffective resistance of trastuzumab to certain patients.

The antibody of the present invention has been genetically engineered to knock out the FUT8 gene, completely eliminating fucose at the Fc end of the antibody, thereby enhancing the ADCC effect of monoclonal antibody drugs. In addition, the anti-HER2 antibody of the invention is completely defucosylated, effectively improves the affinity between the antibody and the Fc receptor CD16a, and improves the ADCC activity of the antibody, the ADCC activity is 20 times that of trastuzumab, and has excellent effects in the treatment of biliary system cancer, gastric cancer, and breast cancer, showing excellent anti-tumor activity, good safety, and tolerance.

### SUMMARY

The present invention provides the use of an anti-HER2 antibody in the preparation of a drug for treating biliary system cancer, gastric cancer, or breast cancer.

The present invention also provides the use of the anti-HER2 antibody in the treatment of biliary system cancer, gastric cancer, or breast cancer.

In some embodiments, the biliary system cancer is cholangiocarcinoma or gallbladder cancer.

In some embodiments, the cholangiocarcinoma is intrahepatic cholangiocarcinoma, extrahepatic cholangiocarcinoma, hilar cholangiocarcinoma, perihilar cholangiocarcinoma, or distal cholangiocarcinoma.

In some embodiments, the cholangiocarcinoma is primary cholangiocarcinoma, recurrent cholangiocarcinoma, or metastatic cholangiocarcinoma.

In some embodiments, the gallbladder cancer is papillary carcinoma, mucinous carcinoma, squamous cell carcinoma, adenosquamous cell carcinoma, or undifferentiated carcinoma.

In some embodiments, the gallbladder cancer is primary gallbladder cancer, recurrent gallbladder cancer, or metastatic gallbladder cancer.

In some embodiments, the gastric cancer is gastric fundus and cardia cancer, gastric body cancer, or gastric antrum cancer.

In some embodiments, the gastric cancer is primary gastric cancer, recurrent gastric cancer, or metastatic gastric cancer.

In some embodiments, the breast cancer is invasive breast cancer, early invasive breast cancer or invasive non-specific breast cancer.

In some embodiments, the breast cancer is an advanced breast cancer.

In some embodiments, the anti-HER2 antibody is a defucosylated anti-HER2 antibody.

In some embodiments, the light chain variable region of the anti-HER2 antibody comprises LCDR1, LCDR2, and LCDR3 as shown in SEQ ID NO: 1, SEQ ID NO: 2, and SEQ ID NO: 3, respectively; and the heavy chain variable region of the anti-HER2 antibody comprises HCDR1, HCDR2, and HCDR3 as shown in SEQ ID NO: 4, SEQ ID NO: 5, and SEQ ID NO: 6, respectively.

In some embodiments, the anti-HER2 antibody is a humanized antibody.

In some embodiments, the light chain variable region sequence of the humanized antibody is as shown in SEQ ID NO: 7, and the heavy chain variable region sequence of the humanized antibody is as shown in SEQ ID NO: 8.

In some embodiments, the use of the anti-HER2 antibody in the preparation of a drug for treating biliary system cancer or in the treatment of biliary system cancer, wherein the dose of the anti-HER2 antibody is 10-20 mg/kg, preferably, the dose of the anti-HER2 antibody is 15 mg/kg.

In some embodiments, the use of the anti-HER2 antibody in the preparation of a drug for treating gastric cancer or in the treatment of gastric cancer, wherein the dose of the anti-HER2 antibody is 5-25 mg/kg, preferably, the dose of the anti-HER2 antibody is 10 mg/kg or 20 mg/kg.

In some embodiments, the use of the anti-HER2 antibody in the preparation of a drug for treating breast cancer or in the treatment of breast cancer, wherein the dose of the anti-HER2 antibody is 5-25 mg/kg, preferably, the dose of the anti-HER2 antibody is 10 mg/kg or 20 mg/kg.

### Detailed Explanation of Terms

In order to facilitate the understanding of the present invention, certain technical and scientific terms are specifically defined below. Unless otherwise explicitly defined in the present invention, all other technical and scientific terms used in this present invention have meanings commonly understood by those skilled in the art to which the present invention belongs.

The term “antibody” refers to natural immunoglobulin or immunoglobulin prepared through partial or complete synthesis. Antibodies can be reconstructed and isolated from natural resources such as plasma or serum, or from the culture supernatant of hybridoma cells that produce antibodies, animal immune serum, and phage library screening. Alternatively, partial or complete synthesis can be achieved through techniques such as genetic recombination. The term “antibody” includes immunoglobulin molecules consisting of four polypeptide chains, two heavy (H) chains, and two light (L) chains interconnected by disulfide bonds, as well as their oligomers (such as IgM). Each L chain is connected to the H chain through a covalent disulfide bond, while two H chains are connected to each other through one or more disulfide bonds depending on the type of H chain. Each heavy chain has a variable region (abbreviated as VH in this article) at the N-terminus, followed by a constant region. Each heavy chain contains a heavy chain variable region (abbreviated as HCVR or VH in the text) and a heavy chain constant region. This heavy chain constant region contains three domains (structural domains), CH1, CH2, and CH3. Each light chain contains a light chain variable region (abbreviated as LCVR or VL in the text) and a light chain constant region. The light chain constant region contains a region (structural domain, CL1). The VH and VL regions can be further subdivided into high variability regions, known as complementary determining regions (CDRs), interspersed with more conservative regions, known as framework regions (FR, also known as skeleton regions or framework regions). Each VH and VL is composed of three CDRs and four FRs, arranged from the amino end to the carboxyl end in the following order: FR1, CDR1, FR2, CDR2, FR3, CDR3, FR4. Antibodies can be antibodies of different subclasses. The term “antibody” includes but is not limited to monoclonal antibodies, whole human antibodies, humanized antibodies, camel antibodies, chimeric antibodies, bispecific or multispecific antibodies, anti-idiotypic (anti Id) antibodies (including, for example, anti Id antibodies against the antibodies disclosed herein), or fusion proteins. These antibodies can belong to any isotype/type (e.g. IgG, IgE, IgM, IgD, IgA, and IgY) or subclass (e.g. IgG1, IgG2, IgG3, IgG4, IgA1, and IgA2).

The term “humanized antibody” refers to chimeric antibodies containing amino acid residues derived from human antibody sequences. Humanized antibodies may contain some or all of the CDRs or HVRs from non-human animals or synthetic antibodies, while the framework and constant regions of the antibody contain amino acid residues derived from the human antibody sequence. It can overcome the heterologous reactions induced by chimeric antibodies due to carrying a large amount of heterologous protein components. This type of structural sequence can be obtained from public DNA databases or publicly available references, including germline antibody gene sequences. To avoid a decrease in activity caused by a decrease in immunogenicity, the human antibody variable region framework sequence can be subjected to minimal reverse or reverse mutations to maintain activity.

The term “variable region” of an antibody refers to the variable region (VL) of a single or combined antibody light chain or variable region (VH) of a single or combined antibody heavy chain. As known in this field, the variable regions of heavy and light chains are each composed of four framework regions (FRs) connected by three complementary determining regions (CDRs) (also known as hypervariable regions). The CDRs in each chain are tightly held together by FR and, together with CDRs from another chain, facilitate the formation of the antigen binding site of the antibody.

The term “variable” refers to the fact that certain segments of the variable domain differ widely in sequence between antibodies. The V domain mediates antigen binding and limits the specificity of specific antibodies to their specific antigens. However, variability is not uniformly distributed throughout the entire variable domain. On the contrary, it is concentrated in three segments called high variability regions (HVR) within the variable domains of light and heavy chains. The more highly conservative part of the variable domain is called the frame region (FR). The variable domains of natural heavy and light chains each contain four FR regions, most of which adopt a β-folding configuration, connected by three HVRs, forming a loop connection and in some cases forming part of the β-folding structure. The HVRs in each chain are tightly held together through the FR region, and together with the HVRs in other chains, promote the formation of antigen binding sites for antibodies. The constant domain is not directly involved in the binding of antibodies and antigens, but exhibits various effector functions, such as participating in antibody dependent cytotoxicity of antibodies.

There are various methods/systems in this field to define and describe CDR, which have been developed and refined for many years, including Kabat, Chothia, IMGT, AbM, and Contact. Kabat is the most commonly used definition of CDR based on sequence variability; Chothia defines CDRs based on the position of structural cyclic regions and sequence variability; the IMGT system defines CDRs based on sequence variability and position within the variable domain structure; AbM is defined based on Oxford Molecular's AbM antibody modeling software, which is a compromise between Kabat and Chothia; Contact defines CDR based on the analysis of complex crystal structures, which is similar to Chothia in multiple aspects. The CDRs listed in the present invention are labeled using the Kabat numbering system.

The term “defucosylated” refers to the process of removing fucoidan from glycoproteins.

The term “chemotherapy” typically refers to the administration of one or more chemotherapy drugs and/or other agents to cancer patients through various methods, including intravenous, oral, intramuscular, intraperitoneal, bladder, subcutaneous, transdermal, or inhalation, or in the form of suppositories.

The term “treatment” refers to a method of obtaining beneficial or desired outcomes (including but not limited to therapeutic and/or preventive benefits). The therapeutic benefit usually refers to the eradication or reduction of the severity of the underlying condition being treated. In addition, therapeutic benefits can be achieved by eradicating, reducing the severity, or decreasing the incidence of one or more physiological symptoms associated with the underlying condition, in order to observe improvement in the subjects (although they may still suffer from the underlying condition). For preventive benefits, the combination can be administered to subjects at risk of developing a specific disease, or to subjects reporting one or more physiological symptoms of the disease, even if the diagnosis of the disease may not have been made.

The term “administration” refers to the act of delivering or causing the delivery of a treatment or drug combination to a subject's body through the methods described in the present invention or as known in the art. The administration of therapeutic or pharmaceutical compositions includes prescribing therapeutic or pharmaceutical compositions to be delivered into the patient's body. Exemplary forms of administration include oral dosage forms such as tablets, capsules, syrups, suspensions; injectable forms, such as intravenous (IV), intramuscular (IM), or intraperitoneal (IP); transdermal formulations, including creams, jellies, powders, or patches; oral dosage form; inhalation powder, spray, suspension and rectal suppository.

The term “subject” typically refers to humans or non-human animals, including but not limited to cats, dogs, horses, pigs, cows, sheep, goats, rabbits, mice, rats, or monkeys. In the present invention, the subject refers to human beings.

The term “dose” refers to the total amount of antibodies administered to a subject during a course of treatment. The dose can be calculated based on weight, such as the amount administered to a subject based on their body weight, expressed in mg/kg.

### DETAILED DESCRIPTION

### Example 1 Preparation of the Test Drug

The heavy chain of the anti-HER2 antibody is as shown in SEQ ID NO: 9, and the light chain is as shown in SEQ ID NO: 10. According to the method for knocking out the FUT8 gene disclosed in patent CN114457110A, using CRISPR/Cas9 technology, a pair of sgRNAs were designed for the exon1 and exon7 regions of the FUT8 gene in CHO cells, and plasmid 1 and plasmid 2 were constructed for the vector in which the FUT8 gene was knocked out; plasmid 1 and plasmid 2 were transfected into CHO cells, the FUT8 gene in CHO cells was knocked out, and stable engineered cells with FUT8 gene silencing were obtained, and then the fully defucosylated anti-HER2 antibody of the present invention was obtained.

Each batch of antibodies produced for clinical purposes meets the requirements of virus safety and the requirements of the Chinese Pharmacopoeia for asepticity. Each batch meets the requirements for characteristics, purity, and effectiveness.

The defucosylated anti-HER2 antibody of the present invention is prepared into a formulation comprising: 20 mg/mL of the defucosylated anti-HER2 antibody, 0.5 mg/ml of histidine hydrochloride monohydrate (pH 6.0), 0.3 mg/mL of histidine, 0.1 mg/ml of polysorbate 20, and 19 mg/mL of trehalose dihydrate (α, α). Each borosilicate penicillin bottle (10 mL) contains 100 mg of the defucosylated anti-HER2 antibody at a concentration of 20 mg/mL.

### Example 2 Phase I Clinical Study of the Anti-HER2 Antibody in Patients with Biliary System Cancer

### 1. Inclusion Criteria

The subjects must meet the following criteria to participate in this study:
(1) The age is 18~75 years old (including the upper and lower limits), and both Chinese men and women can participate.
(2) Phase I study: HER2 positive advanced solid tumor subjects diagnosed by histopathology and/or cytology who have failed standard treatment. Standard treatment failure refers to disease progression during or after the last treatment, inability to tolerate toxic side effects during treatment (hematological toxicity≥Grade 4 or non-hematological toxicity≥Grade 3 after previous standard treatment), disease progression or recurrence during neoadjuvant/adjuvant therapy or within 6 months after the end of treatment. HER2 positivity is defined as IHC 3+, or IHC 2+ and positive/HER2 gene amplification detected by fluorescence in situ hybridization (FISH).
(3) According to RECIST 1.1 standard, there must be at least one measurable lesion that has not received local treatment (including local radiotherapy, ablation, and interventional therapy, etc.).
(4) The ECOG score for physical condition ranges from 0 to 1.
(5) Having good organ function (the upper limit of the normal value is based on the normal range of each research center), if white blood boosting drugs, thrombopoietin, and colony-stimulating factor have been received before the examination, they should be washed for at least one week. Laboratory tests should meet the following requirements: \textcircled{1} Blood routine: hemoglobin (HGB)≥100 g/L, white blood cell count (WBC)≥3.0×10⁹/L, neutrophil count (ANC)≥1.5×10⁹/L, platelet count (PLT)≥75×10⁹/L; \textcircled{2} Blood biochemistry: total bilirubin (TBIL)≤1.5×upper limit of normal (ULN), alanine aminotransferase (ALT) and aspartate aminotransferase (AST)≤3.0×ULN, serum creatinine (Cr)≤1.5×ULN, or creatinine clearance rate≥50 mL/min calculated according to the Cockcroft-Gault formula. For subjects with liver cancer or liver metastasis, ALT and AST≤5.0×ULN, and TBIL≤3.0×ULN.
(6) Left ventricular ejection fraction (LVEF)≥50%.
(7) Expected survival period≥3 months.
(8) The subjects must agree to use at least one medically approved contraceptive measure (female: such as intrauterine devices, birth control pills or condoms; male: such as condoms, abstinence, etc.) during the study treatment period and for 6 months after the end of the study treatment period. Female subjects must be non-lactating period.
(9) The subjects should be fully informed of the research content, process, possible risks and benefits and sign the informed consent. The subjects should have good compliance and be able to cooperate to complete the study and follow-up.

### 2. Efficacy Evaluation Criteria

The effectiveness of this study is exploratory observation, with RECIST 1.1 as the evaluation criterion, and tumor imaging evaluation is conducted every 2 cycles (±7 days). After 6 months of the first medication, tumor imaging evaluation should be conducted every 4 cycles (±7 days) as appropriate.

The assessment of tumor remission will include all known or suspected sites of onset. Imaging includes computed tomography (CT) or magnetic resonance imaging (MRI) scans of the brain, neck, chest, abdomen, and pelvic cavity, bone scans, and/or bone X-rays for patients with known or suspected bone metastases.

In subsequent tumor assessment, the same imaging techniques should be used for lesions of the same type as during the screening period. The evaluation of anti-tumor activity will be conducted through radiographic imaging during the screening period and treatment process according to the experimental flowchart. When there is suspicion of disease progression (such as worsening symptoms) and the subject withdraws from treatment (if no evaluation has been conducted within the previous 28 days), evaluation should also be conducted.

### 3. Treatment Overview

Two subjects were enrolled in the study, and the observed safety events were consistent with the characteristics of the biliary system cancer patient population. No new safety signals were observed. One patient underwent tumor evaluation after at least one treatment, and one patient achieved partial response (PR) with a disease control rate (DCR) of 100% and an objective response rate (ORR) of 100%. The clinical trial results show that the defucosylated anti-HER2 antibody exhibits good anti-tumor effect.

### 4. Typical Cases

Previous tumor treatment history of this case: \textcircled{1} Protocol/drug name: albumin-bound paclitaxel, capecitabine; \textcircled{2} Treatment method: chemotherapy; \textcircled{3} Best therapeutic effect: PR (partial response).

Dose: 15 mg/kg.

Administration cycle: every three weeks as a cycle, with administration.

Therapeutic efficacy: the efficacy was evaluated in the second cycle as SD; the efficacy was evaluated in the fourth and sixth cycles as PR, confirming PR. The efficacy was evaluated in the eighth cycle as PD (progressive disease), i.e. DOR (duration of remission) for 4 cycles, 12 weeks.

Adverse reactions: CTCAE Grade 1 adverse reactions such as anemia, hyperuricemia, diarrhea, constipation, and elevated alanine aminotransferase.

The results show that the defucosylated anti-HER2 antibody of the present invention exhibits excellent anti-tumor activity, as well as good safety and tolerability.

### Example 3 Phase I Clinical Study of the Anti-HER2 Antibody in Patients with Gastric Cancer

### 1. Inclusion Criteria

The subjects must meet the following criteria to participate in this study:
(1) The age is 18~75 years old (including the upper and lower limits), and both Chinese men and women can participate.
(2) Phase I study: HER2 positive advanced solid tumor subjects diagnosed by histopathology and/or cytology who have failed standard treatment. Standard treatment failure refers to disease progression during or after the last treatment, inability to tolerate toxic side effects during treatment (hematological toxicity≥Grade 4 or non-hematological toxicity≥Grade 3 after previous standard treatment), disease progression or recurrence during neoadjuvant/adjuvant therapy or within 6 months after the end of treatment. HER2 positivity is defined as IHC 3+, or IHC 2+ and positive/HER2 gene amplification detected by fluorescence in situ hybridization (FISH).
(3) According to RECIST 1.1 standard, there must be at least one measurable lesion that has not received local treatment (including local radiotherapy, ablation, and interventional therapy, etc.).
(4) The ECOG score for physical condition ranges from 0 to 1.
(5) Having good organ function (the upper limit of the normal value is based on the normal range of each research center), if white blood boosting drugs, thrombopoietin, and colony-stimulating factor have been received before the examination, they should be washed for at least one week. Laboratory tests should meet the following requirements: \textcircled{1} Blood routine: hemoglobin (HGB)≥100 g/L, white blood cell count (WBC)≥3.0×10⁹/L, neutrophil count (ANC)≥1.5×10⁹/L, platelet count (PLT)≥75×10⁹/L; \textcircled{2} Blood biochemistry: total bilirubin (TBIL)≤1.5×upper limit of normal (ULN), alanine aminotransferase (ALT) and aspartate aminotransferase (AST)≤3.0×ULN, serum creatinine (Cr)≤1.5×ULN, or creatinine clearance rate≥50 mL/min calculated according to the Cockcroft-Gault formula. For subjects with liver cancer or liver metastasis, ALT and AST≤5.0×ULN, and TBIL≤3.0×ULN.
(6) Left ventricular ejection fraction (LVEF)≥50%.
(7) Expected survival period≥3 months.
(8) The subjects must agree to use at least one medically approved contraceptive measure (female: such as intrauterine devices, birth control pills or condoms; male: such as condoms, abstinence, etc.) during the study treatment period and for 6 months after the end of the study treatment period. Female subjects must be non-lactating period.
(9) The subjects should be fully informed of the research content, process, possible risks and benefits and sign the informed consent. The subjects should have good compliance and be able to cooperate to complete the study and follow-up.

### 2. Therapeutic Efficacy Evaluation Criteria

The effectiveness of this study is exploratory observation, with RECIST 1.1 as the evaluation criterion, and tumor imaging evaluation is conducted every 2 cycles (±7 days). After 6 months of the first medication, tumor imaging evaluation should be conducted every 4 cycles (±7 days) as appropriate.

The assessment of tumor remission will include all known or suspected sites of onset. Imaging includes computed tomography (CT) or magnetic resonance imaging (MRI) scans of the brain, neck, chest, abdomen, and pelvic cavity, bone scans, and/or bone X-rays for patients with known or suspected bone metastases.

In subsequent tumor assessment, the same imaging techniques should be used for lesions of the same type as during the screening period. The evaluation of anti-tumor activity will be conducted through radiographic imaging during the screening period and treatment process according to the experimental flowchart. When there is suspicion of disease progression (such as worsening symptoms) and the subject withdraws from treatment (if no evaluation has been conducted within the previous 28 days), evaluation should also be conducted.

### 3. Treatment Overview

Two subjects were enrolled in the study, and the observed safety events were consistent with the characteristics of the gastric cancer patient population. No new safety signals were observed. Two of the patients underwent tumor evaluation after at least one treatment, and their condition was stable disease (SD), the disease control rate (DCR) was 100%. The clinical trial results show that the defucosylated anti-HER2 antibody exhibits good anti-tumor effect.

### 4. Typical Cases

### Case 1

Previous tumor treatment history of this case: \textcircled{1} Protocol/drug name: oxaliplatin, capecitabine, trastuzumab, irinotecan, apatinib, SHR-1701, albumin-bound paclitaxel; \textcircled{2} Treatment methods: chemotherapy, targeted therapy, immunotherapy; \textcircled{3} Best therapeutic effect: PR.

Dose: 10 mg/kg.

Administration cycle: every three weeks as a cycle, with administration.

Therapeutic efficacy: the efficacy was conducted in the 2nd, 4th, 6th, and 8th cycles, all of which were SD (stable disease).

Adverse reactions: hypocalcemia, hypoalbuminemia, hyperuricemia, anemia, among which anemia is a CTCAE Grade 2 adverse reaction, and the rest are CTCAE Grade 1 adverse reactions.

### Case 2

Previous tumor treatment history of this case: \textcircled{1} Protocol/drug name: trastuzumab, oxaliplatin, capecitabine, tegafur, albumin-bound paclitaxel; \textcircled{2} Treatment methods: targeted therapy, chemotherapy; \textcircled{3} Best therapeutic effect: PR.

Dose: 20 mg/kg.

Administration cycle: every three weeks as a cycle, with administration.

Therapeutic efficacy: the efficacy was evaluated in cycles 2 and 4, both of which were SD.

Adverse reactions: CTCAE Grade 1 adverse reactions such as hypoalbuminemia, phlebitis, and urinary tract infections.

The results show that the defucosylated anti-HER2 antibody of the present invention exhibits excellent anti-tumor activity, as well as good safety and tolerability.

### Example 4 Phase I Clinical Study of the Anti-HER2 Antibody in Patients with Breast Cancer

### 1. Inclusion Criteria

The subjects must meet the following criteria to participate in this study:
(1) The age is 18~75 years old (including the upper and lower limits), and both Chinese men and women can participate.
(2) Phase I study: HER2 positive advanced solid tumor subjects diagnosed by histopathology and/or cytology who have failed standard treatment. Standard treatment failure refers to disease progression during or after the last treatment, inability to tolerate toxic side effects during treatment (hematological toxicity≥Grade 4 or non-hematological toxicity≥Grade 3 after previous standard treatment), disease progression or recurrence during neoadjuvant/adjuvant therapy or within 6 months after the end of treatment. HER2 positivity is defined as IHC 3+, or IHC 2+ and positive/HER2 gene amplification detected by fluorescence in situ hybridization (FISH).
(3) According to RECIST 1.1 standard, there must be at least one measurable lesion that has not received local treatment (including local radiotherapy, ablation, and interventional therapy, etc.).
(4) The ECOG score for physical condition ranges from 0 to 1.
(5) Having good organ function (the upper limit of the normal value is based on the normal range of each research center), if white blood boosting drugs, thrombopoietin, and colony-stimulating factor have been received before the examination, they should be washed for at least one week. Laboratory tests should meet the following requirements: \textcircled{1} Blood routine: hemoglobin (HGB)≥100 g/L, white blood cell count (WBC)≥3.0×10⁹/L, neutrophil count (ANC)≥1.5×10⁹/L, platelet count (PLT)≥75×10⁹/L; \textcircled{2} Blood biochemistry: total bilirubin (TBIL)≤1.5×upper limit of normal (ULN), alanine aminotransferase (ALT) and aspartate aminotransferase (AST)≤3.0×ULN, serum creatinine (Cr)≤1.5×ULN, or creatinine clearance rate≥50 mL/min calculated according to the Cockcroft-Gault formula. For subjects with liver cancer or liver metastasis, ALT and AST≤5.0×ULN, and TBIL≤3.0×ULN.
(6) Left ventricular ejection fraction (LVEF)≥50%.
(7) Expected survival period≥3 months.
(8) The subjects must agree to use at least one medically approved contraceptive measure (female: such as intrauterine devices, birth control pills or condoms; male: such as condoms, abstinence, etc.) during the study treatment period and for 6 months after the end of the study treatment period. Female subjects must be non-lactating period.
(9) The subjects should be fully informed of the research content, process, possible risks and benefits and sign the informed consent. The subjects should have good compliance and be able to cooperate to complete the study and follow-up.

### 2. Therapeutic Efficacy Evaluation Criteria

The effectiveness of this study is exploratory observation, with RECIST 1.1 as the evaluation criterion, and tumor imaging evaluation is conducted every 2 cycles (±7 days). After 6 months of the first medication, tumor imaging evaluation should be conducted every 4 cycles (±7 days) as appropriate.

The assessment of tumor remission will include all known or suspected sites of onset. Imaging includes computed tomography (CT) or magnetic resonance imaging (MRI) scans of the brain, neck, chest, abdomen, and pelvic cavity, bone scans, and/or bone X-rays for patients with known or suspected bone metastases.

In subsequent tumor assessment, the same imaging techniques should be used for lesions of the same type as during the screening period. The evaluation of anti-tumor activity will be conducted through radiographic imaging during the screening period and treatment process according to the experimental flowchart. When there is suspicion of disease progression (such as worsening symptoms) and the subject withdraws from treatment (if no evaluation has been conducted within the previous 28 days), evaluation should also be conducted.

### 3. Treatment Overview

A total of 8 subjects were enrolled in the group. The observed safety events were consistent with the characteristics of breast cancer patients, and no new safety signals were observed. Among them, 7 patients underwent tumor evaluation after at least one treatment, and 2 patients had stable disease (SD) conditions with a disease control rate (DCR) of 28.6%. The clinical trial results show that the defucosylated anti-HER2 antibody exhibits good anti-tumor effect.

### 4. Typical Cases

### Case 1

Previous tumor treatment history of this case: \textcircled{1} Protocol/drug name: docetaxel, trastuzumab, pertuzumab; \textcircled{2} Treatment methods: chemotherapy, targeted therapy; \textcircled{3} Best therapeutic effect: PR.

Dose: 10 mg/kg.

Administration cycle: every three weeks as a cycle, with administration.

Therapeutic efficacy: the efficacy was conducted in the 2nd, 4th, 6th, 8th, 10th, 12th, 14th, and 16th cycles, all of which were SD (stable disease).

Adverse reactions: CTCAE Grade 1 adverse reactions such as hypoalbuminemia, hyperglycemia, leukopenia, and neutropenia.

### Case 2

Previous tumor treatment history of this case: \textcircled{1} Protocol/drug name: pyrotinib tablets/placebo, trastuzumab, docetaxel; \textcircled{2} Treatment methods: targeted therapy, chemotherapy; \textcircled{3} Best therapeutic effect: PR.

Dose: 20 mg/kg.

Administration cycle: every three weeks as a cycle, with administration.

Therapeutic efficacy: evaluate the efficacy in the second cycle as SD.

Adverse reactions: Grade 1 CTCAE adverse reactions such as hypoalbuminemia, hypocalcemia, and elevated alanine aminotransferase.

The results show that the defucosylated anti-HER2 antibody of the present invention exhibits excellent anti-tumor activity, as well as good safety and tolerability.

The protected content of the present invention is not limited to the above embodiments. All variations and advantages that can be conceived by those skilled in the art without departing from the spirit and scope of the inventive concept are included in the present invention and are protected by the appended claims.

1. Use of an anti-HER2 antibody in the preparation of a drug for treating biliary system cancer, gastric cancer, or breast cancer.

2. The use of the anti-HER2 antibody of claim 1, wherein the biliary system cancer is cholangiocarcinoma or gallbladder cancer.

3. The use of the anti-HER2 antibody of claim 2, wherein the cholangiocarcinoma is intrahepatic cholangiocarcinoma, extrahepatic cholangiocarcinoma, hilar cholangiocarcinoma, perihilar cholangiocarcinoma, or distal cholangiocarcinoma.

4. The use of the anti-HER2 antibody of claim 3, wherein the cholangiocarcinoma is primary cholangiocarcinoma, recurrent cholangiocarcinoma, or metastatic cholangiocarcinoma.

5. The use of the anti-HER2 antibody of claim 2, wherein the gallbladder cancer is papillary carcinoma, mucinous carcinoma, squamous cell carcinoma, adenosquamous carcinoma, or undifferentiated carcinoma.

6. The use of the anti-HER2 antibody of claim 5, wherein the gallbladder cancer is primary gallbladder cancer, recurrent gallbladder cancer, or metastatic gallbladder cancer.

7. The use of the anti-HER2 antibody of claim 1, wherein the gastric cancer is gastric fundus and cardia cancer, gastric body cancer, or gastric antrum cancer.

8. The use of the anti-HER2 antibody of claim 7, wherein the gastric cancer is primary gastric cancer, recurrent gastric cancer, or metastatic gastric cancer.

9. The use of the anti-HER2 antibody of claim 1, wherein the breast cancer is invasive breast cancer, early invasive breast cancer or invasive non-specific breast cancer.

10. The use of the anti-HER2 antibody of claim 9, wherein the breast cancer is an advanced breast cancer.

11. The use of the anti-HER2 antibody of claim 1, wherein the anti-HER2 antibody is a defucosylated anti-HER2 antibody.

12. The use of claim 11, wherein the light chain variable region of the anti-HER2 antibody comprises LCDR1, LCDR2, and LCDR3 as shown in SEQ ID NO: 1, SEQ ID NO: 2, and SEQ ID NO: 3, respectively; and the heavy chain variable region of the anti-HER2 antibody comprises HCDR1, HCDR2, and HCDR3 as shown in SEQ ID NO: 4, SEQ ID NO: 5, and SEQ ID NO: 6, respectively.

13. The use of the anti-HER2 antibody of claim 12, wherein the anti-HER2 antibody is a humanized antibody.

14. The use of the anti-HER2 antibody of claim 13, wherein the light chain variable region sequence of the humanized antibody is as shown in SEQ ID NO: 7, and the heavy chain variable region sequence of the humanized antibody is as shown in SEQ ID NO: 8.

15. Use of the anti-HER2 antibody of claim 2, wherein the dose of the anti-HER2 antibody is 10-20 mg/kg.

16. The use of the anti-HER2 antibody of claim 8, wherein the dose of the anti-HER2 antibody is 5-25 mg/kg.

17. The use of the anti-HER2 antibody of claim 10, wherein the dose of the anti-HER2 antibody is 5-25 mg/kg.
